(19) United States
(12) Patent Application Publication
(10) Pub. No.: US 2014/0273016 A1
(43) Pub. Date: Sep. 18, 2014

(54) DEVICE AND METHOD FOR THE DETECTION AND DIAGNOSIS OF AGGRESSIVE AND METASTATIC CANCER AND CANCER STEM CELLS EMPLOYING PODOCALYXIN AND TRA BIOMARKERS

(71) Applicant: WILLIAM MICHAEL SCHOPPERLE, South Boston, MA (US)

(72) Inventor: WILLIAM MICHAEL SCHOPPERLE, South Boston, MA (US)

(21) Appl. No.: 14/206,434

(22) Filed: Mar. 12, 2014

Related U.S. Application Data
(60) Provisional application No. 61/781,992, filed on Mar. 14, 2013.
(60) Provisional application No. 61/782,147, filed on Mar. 14, 2013.

(51) Int. Cl. G01N 33/574

(52) U.S. Cl. CPC G01N 33/57492
USPC 435/7.23; 435/287.2

(57) ABSTRACT

The invention relates to devices and methods to detect podocalyxin and TRA molecules in a biological sample obtained from a patient and therefore, to detect the presence of podocalyxin-expressing and TRA-expressing cancer stem cells in a patient. The invention further relates to devices and methods to determine a diagnosis of aggressive or metastatic cancer by employing podocalyxin and TRA as biomarkers.

### CROSS REFERENCE TO RELATED PATENT APPLICATIONS

This patent application claims the benefit of priority under 35 U.S.C. §119(e) from U.S. Provisional Patent Application No. 61/781,992 entitled “System and Method for the Detection and Diagnosis of Aggressive and Metastatic Cancer Employing Podocalyxin Biomarker”, filed on Mar. 14, 2013; and U.S. Provisional Patent Application No. 61/782,147 entitled “Device and Method for the Detection and Diagnosis of Aggressive and Metastatic Cancer and Cancer Stem Cells”, filed on Mar. 14, 2013; which are incorporated in their entirety herein by reference.

### 1. FIELD OF THE INVENTION

The invention generally relates to a device and method to detect the presence of podocalyxin and/or TRA, such as TRA-1-60 and TRA-1-81 epitopes, in a biological sample to detect the presence of podocalyxin-expressing and/or TRA-expressing cancer stem cells or cancer cells with stem cell-like properties in a patient, and to determine a diagnosis of aggressive and/or metastatic cancer by employing the podocalyxin and/or TRA as biomarker.

### 2. BACKGROUND OF THE INVENTION

Human cancer can be broadly categorized into two general types, i.e., localized cancer and aggressive cancer. It is generally known that localized cancer does not spread, is non-lethal and, is usually manageable and treatable with known conventional cancer therapies. In contrast, aggressive cancer typically spreads beyond the site of the primary tumor to other parts of the body, is lethal and usually is not curable with known conventional cancer therapies and is almost always lethal for the patient. It is believed that localized cancer cells transform into aggressive cancer cells and acquire specialized cell functions allowing them to detach from the primary tumor, move through surrounding tissues and into the blood supply and then, to reattach and seed new tumor growth elsewhere in the body.

There are many patients that are diagnosed with localized cancer tumors. The cancer tumors may be detected based on the presence of a mass in a patient. The diagnosis of cancer based on the presence of the mass, i.e., the tumor itself, may result in a late stage diagnosis. Further, cancer diagnosis based on the presence of the mass may not provide information as to whether the cancer cells are localized or aggressive, i.e., a metastatic stage.

It is often difficult to identify and diagnose cancer at a pre-invasive stage because the carcinoma can be asymptomatic. The diagnosis is typically provided following a surgical biopsy and this diagnostic procedure is generally only carried out in patients at-risk or following a clinical examination and/or ultrasonography with a suspicious outcome. The diagnostic procedures currently available include surgical biopsy which is most often performed as an open incision of a tissue sample and sometimes as a needle-biopsy. After proper histological processing, the sample is examined by a pathologist. The finding of cancer cells will typically be treated by irradiation and sometimes with adjuvant chemotherapy.

Known diagnostic procedures can be expensive, time-consuming and invasive, and may result in serious complications. Further, these known procedures may not be capable of determining if the cancer that is detected is of an aggressive or metastatic form.

The time course for progression of localized cancer to lethal aggressive or metastatic cancer has significant variability. Some cancers progress rapidly while others remain indolent for years prior to progressing to and aggressive or metastatic form, and some cancers will never progress to an aggressive or metastatic form. It is not known why some cancers progress rapidly while others progress slowly and still others do not progress at all. For example, it is estimated that only a small percentage, such as less than 10%, of localized prostate cancer tumors will develop into an aggressive, metastatic stage.

Known cancer treatment can be less effective or non-effective against aggressive or metastatic cancer cells and therefore, these cancer cells can survive the cancer treatment and can re-seed the cancer causing new tumor growth following administration of the treatment. It would be beneficial to determine which patients having localized cancer will develop aggressive or metastatic cancer and which patients will not proceed beyond the localized stage. Thus, those patients that are known not to develop the aggressive disease can receive treatment for the localized cancer only and those that are determined as proceeding beyond the localized cancer can receive early treatment for the aggressive or metastatic cancer. Thus, the patients without the aggressive disease can be administered a less aggressive treatment regimen, such as simply surveillance and monitoring of the patient's condition and progression, and these patients do not need to undergo aggressive treatments, such as surgery, chemotherapy and radiation therapy, that have serious complications associated therewith. It has, however, proven to be difficult to determine those patients will develop the aggressive disease and those that will develop only localized cancer.

Podocalyxin is a cell surface plasma membrane protein which is expressed in a few types of normal cells and is highly expressed on the surface of human pluripotent stem cells (e.g., cancer cells). Pluripotent stem cells are fundamental stem cells having biological properties to differentiate into any one of the two hundred different kinds of cells that make up the human body. Podocalyxin has several putative cellular functions in normal cells which include a cellular adhesion function, an anti-adhesion protein and a cell motility function. Podocalyxin can be cleaved or clipped-off the surface of a cancer cell by a biological process to generate a soluble form that can enter the blood or other biological fluids of a patient. Thus, although the exact function of podocalyxin in cancer has yet to be determined, these functions of podocalyxin may allow cancer cells to survive within a patient and furthermore, to spread or metastasize.

In the art, it is known that a TRA molecule (also referred to as an epitope) is a carbohydrate (sugar) molecular structure which is present in large amounts on the cell surface of undifferentiated human embryonic pluripotent stem cells. This carbohydrate molecule was first discovered several decades ago on human embryonal carcinoma pluripotent cancer stem cells by using two distinct mouse monoclonal antibodies named TRA-1-60 and TRA-1-81. At the time of discovery, the epitopes to which these monoclonal antibodies bind was unknown and therefore, the epitopes were named after the antibodies TRA-1-60 and TRA-1-81. An epitope is the molecular structure which a particular antibody specifically reacts with and to which it attaches or binds. These antibodies are widely used in stem cell research because the TRA-1-60 and TRA-1-81 epitopes are human biomarkers for pluripotent embryonic stem cells. All pluripotent stem cells, regardless of how they are generated, have the TRA-1-60/TRA-1-81 carbohydrate structure on the surface of their cells. If TRA is not present, the cells are not stem cells.

The TRA epitope carbohydrate structure has been identified as a pluripotency associated type 1 lactosamine epitope. It has also been reported that the minimal epitope necessary for binding of the TRA-1-60 and TRA-1-81 antibodies is Galβ1-3GlcNAcβ1-3Galβ1-4GlcNAc on an extended tetrasaccharide mucin type O-glycan structure in human embryonic stem cells with a likely structure Galβ1-3GlcNAcβ1-3Galβ1-4GlcNAcβ1-6(Galβ1-3)GalNAc.

Podocalyxin is a glycosylated membrane protein with amino acid sequence homology to the hematopoietic stem cell marker CD 34. Two forms of podocalyxin are known to exist. One form is a normal form found in certain subtypes of cells within the body. Another form is a stem-cell version of podocalyxin that has the TRA carbohydrate structures attached to it to form a TRA-podocalyxin molecule. Thus, podocalyxin is the ‘molecular carrier’ of the TRA structures on human stem cells. Purified podocalyxin has binding activity with the TRA-1-60 and TRA-1-81 antibodies. Since podocalyxin can be cleaved or clipped-off the surface of the cell by a biological process to generate a soluble form that can enter the blood or other biological fluids of a patient, and TRA molecules are attached to podocalyxin, the TRA molecules can also detach from the cell and enter the blood or other biological fluids of the patient.

Research studies have identified a small subset of stem cell-like cancer cells and it is believed that these are the cancer cells responsible for causing aggressive metastatic disease in the body, such as the prostate, ovary, colon and breast. These cancer cells have podocalyxin on the surface of their cells. If cancer cells have embryonic properties or pluripotent properties, then these cells can have TRA on the surface of their cells. These podocalyxin- and/or TRA-specific cancer cells make up only a small portion of the tumor bulk, but they have stem cell-like properties and it is believed that these are the cells which can survive current standard cancer treatments. They are highly resistant and motile, can reseed the tumor and cause new growth after treatments.

Despite known means of detecting and diagnosing cancer in a patient, there is lacking an effective means of detecting the presence of aggressive and metastatic cancer cells by the use of a patient biological sample, such as a blood-derived specimen including blood serum or blood plasma, and other biological fluids, such as seminal plasma and urine.

Thus, it is desirous to develop devices and methods for detecting aggressive and/or metastatic cancer cells based on a biological sample from a patient to preclude the need for invasive procedures which are currently employed, to obtain an earlier diagnosis, to determine whether the cancer cells are in the aggressive, e.g., metastatic stage, and to formulate a diagnosis and treatment based on knowing that the aggressive form of the cancer cells are present or absent in the patient.

The devices and methods of the invention include detecting the presence of podocalyxin and/or TRA molecules in a patient by determining the presence or absence of a biomarker, i.e., podocalyxin, TRA-1-60, TRA-1-81, and mixtures thereof, in a patient biological sample, such as a blood-derived specimen or other fluid specimen. Thus, it is an objective of the invention to provide a diagnostic tool to detect in a cancer-containing patient the presence of a subset of aggressive, metastatic and resistant cancer cells that have specialized stem cell-like properties enabling them to survive standard cancer treatments and, to grow and metastasize throughout the body potentially resulting in a lethal form of the disease.

### SUMMARY OF THE INVENTION

In one aspect, the invention provides a method of detecting the presence of podocalyxin and/or TRA molecules in a first patient. The method includes obtaining a first biological sample from the first patient, the first sample selected from the group consisting of a blood-derived specimen, a seminal fluid specimen, a urine specimen, and combinations thereof; testing the first biological sample for the presence of podocalyxin and/or TRA molecules; and determining the presence or absence of podocalyxin and/or TRA molecules in the first biological sample.

The podocalyxin and/or TRA molecules can be selected from the group consisting of anti-podocalyxin, TRA-1-60, TRA-1-81, and mixtures thereof.

The first biological sample can be compared to a second biological sample selected from the group consisting of an earlier obtained biological sample from the first patient or a control biological sample obtained from a second patient without cancer.

The first biological sample can be compared to a predetermined threshold value.

The method can further include providing a medical diagnosis of a presence of metastatic cancer cells in the first patient based on the presence of the podocalyxin and/or TRA molecules in the first biological sample or providing a medical diagnosis of an absence of metastatic cancer in the patient based on the absence of the podocalyxin and/or TRA molecules in the sample.

The method can further include providing a medical diagnosis of a presence of metastatic cancer cells in the first patient based on a level of the podocalyxin and/or TRA molecules in the first biological sample being measurably greater than a level of podocalyxin and/or TRA molecules in the second biological sample or providing a medical diagnosis of an absence of metastatic cancer cells in the first patient based on a level of the podocalyxin and/or TRA molecules in the first biological sample being essentially equal to or measurably less than a level of podocalyxin and/or TRA molecules in the second biological sample.

The method can further include providing a medical diagnosis of a presence of metastatic cancer cells in the first patient based on a level of the podocalyxin and/or TRA molecules in the first biological sample being essentially equal to or measurably greater than the threshold value or providing a medical diagnosis of an absence of metastatic cancer cells in the first patient based on a level of the podocalyxin and/or TRA molecules in the first biological sample being measurably less than the threshold value.

In another aspect, the invention provides a method of detecting and diagnosing aggressive cancer stem cells in a patient. The method includes obtaining a biological sample from the patient, the biological sample selected from the group consisting of a blood-derived specimen, urine specimen and combinations thereof; analyzing the biological sample for a presence of podocalyxin and/or TRA molecules selected from the group consisting of anti-podocalyxin, TRA-1-60, TRA-1-81 and mixtures thereof; determining the presence or an absence of the podocalyxin and/or TRA molecules in the biological sample; providing a diagnosis of the presence of aggressive cancer stem cells in the patient based on the presence of the podocalyxin and/or TRA molecules in the biological sample; and providing a diagnosis of the absence of aggressive cancer stem cells in the patient based on the absence of the podocalyxin and/or TRA molecules in the biological sample.

The analyzing of the biological sample can include employing a binding material selected to interact with the podocalyxin and/or TRA molecules in the biological sample. The binding material can be an antibody.

In yet another aspect, the invention provides a device for detecting and diagnosing aggressive cancer stem cells in a patient. The device includes a tool for obtaining a biological sample from the patient, the biological sample selected from the group consisting of a blood-derived specimen, a seminal fluid specimen, a urine specimen and combinations thereof; a detector means for determining the presence or absence of podocalyxin and/or TRA molecules selected from the group consisting of anti-podocalyxin, TRA-1-60, TRA-1-81 and mixtures thereof; and an indicator means to display the presence of the podocalyxin and/or TRA molecules in the biological sample.

The device can be a test kit which includes an enzyme-linked immunosorbent assay and, at least one testing plate coated with a binding material selected to interact with anti-podocalyxin, TRA-1-60 or TRA-1-81 and to separate the podocalyxin and/or TRA molecules from the biological sample.

### DETAILED DESCRIPTION OF THE INVENTION

The invention relates to biomarkers for use in detecting, diagnosing, measuring and monitoring aggressive and/or metastatic cancer cells in a patient. The invention includes devices and methods for detecting the presence of podocalyxin and/or TRA molecules in a biological sample obtained from a patient. Further, this invention includes devices and methods for determining the presence or absence of podocalyxin, TRA-1-60, TRA-1-81, and mixtures thereof in a blood-derived specimen, such as blood plasma and blood serum, or a seminal fluid specimen, such as seminal plasma, or a urine specimen of a patient. Furthermore, this invention includes devices and methods for detecting and diagnosing aggressive and/or metastatic cancer stem cells or cancer cells having stem cell-like properties in a patient. Moreover, this invention includes devices and methods for providing a medical diagnosis of a patient with aggressive and/or metastatic cancer based on the presence of podocalyxin and/or TRA molecules in the biological sample of the patient. This invention also includes a test kit.

Without intending to be bound by any particular theory, it is believed the stem-cell form of podocalyxin and TRA molecules are embryonic- and pluripotent stem-cell specific structures. Podocalyxin is a cell surface plasma membrane adhesion protein which is highly expressed on the surface of human pluripotent stem cells, e.g., human embryonic stem cells. TRA is an embryonic carbohydrate structure which is present on the cell surface of undifferentiated human embryonic stem cells and human pluripotent stem cells. It is also present on embryonal carcinoma cancer stem cells which are considered to be a malignant version of the normal embryonic stem cells. Podocalyxin and TRA are present on normal stem cells, such as the embryonic stem cells and the like, and cancer stem cells. As used herein, the term “normal” and related variations thereof, refers to the absence of cancer. Thus, podocalyxin and TRA molecules may be found in normal patients and patients having aggressive cancer. As used herein, the term “cancer stem cell” and related variations thereof, means a cancer stem cell with the ability to differentiate into other cell types, or a cancer cell with some properties which are shared with stem cells but the cell may not be able to differentiate into other cell types, or a cell which is not defined as cancer by current medical standards but would have cellular changes which would not be present in a normal cell.

Further, it is believed that the TRA molecule covalently attaches to podocalyxin. The combination of TRA and podocalyxin, TRA-podocalyxin protein, may be found on normal stem cells and cancer stem cells and therefore, may be found in normal patients and patients having aggressive cancer. It is contemplated that podocalyxin, TRA and TRA-podocalyxin may be present in normal patients at low levels and may be present in patients having aggressive and/or metastatic cancers at higher levels.

Furthermore, without intending to be bound by any particular theory, it is believed that aggressive and metastatic forms of cancer are caused by a unique type of cancer cells which have stem cell-like properties. These cancer stem cells can develop in localized cancer tissue or tumors. These cancer stem cells have podocalyxin and/or TRA expressed on their surface. As used herein, the term “expressed” and related variations thereof, means a cell with the podocalyxin and/or TRA molecule present on the surface of the cancer stem cell, or present intracellularly in the cytoplasm or associated with an organelle, or present in the extracellular space and/or the glycocalyx surrounding the cell and not necessarily in direct physical contact with the cancer stem cell.

The podocalyxin expressed on a stem cell can be clipped-off of the surface of the stem cell by a biological process and as a result, generate a soluble form of podocalyxin that can enter into the blood or other biological fluid of a patient. Further, it is believed that podocalyxin can also be found in blood and other biological fluids in an insoluble form, e.g., on the surface of tumor cells which are circulating through the blood or other biological fluids. Since TRA is attached to podocalyxin on the surface of the stem cell, the TRA-podocalyxin protein can be clipped-off of the surface by a biological process and enter into the blood or other biological fluid of the patient. Thus, podocalyxin and/or TRA may be present and detectable in the blood or biological fluid.

The advantages of the devices and methods of the invention over those currently employed for the detection of cancer can include early detection, non-invasive procedure, specific and accurate detection, and the ability to customize a treatment regimen in accordance with the aggressive and metastatic or non-aggressive nature of the cancer stem cells. Since the biological sample of the patient can be collected, tested and evaluated with or without the presence of a tumor or mass, aggressive and/or metastatic cancer cells can be diagnosed prior to the formation of any aggressive and/or metastatic tumors. The biological sample can include various fluids obtained from the patient, such as blood, seminal fluid and urine. As used herein the term “blood” refers to and encompasses a blood-derived specimen such as blood serum and blood plasma, and the term “seminal fluid” encompasses seminal plasma. The devices and methods of the invention rely on the examination and analysis of a patient's biological sample and does not require biopsy or orchiectomy. The analysis of the biological sample is performed in a specific manner to reduce the risk of obtaining false positive and false negative results.

The biological sample can be collected from the patient using a variety of conventional apparatus and techniques known in the art. For example, a blood sample can be withdrawn from the patient using a syringe or the like, and a urine sample can be collected from the patient during urination. The biological sample can be collected in a labeled container or placed in a labeled container. The label of the container may contain a unique identification number and one part of the label may be transferable to a slide. The sample can be collected in a container which may contain stabilizing agents to aid in preservation of the sample and of the signal/marker quality in the sample. Stabilizing agents include but are not limited to pH-buffers, Protease-inhibitors, RNase inhibitors, fixatives and other compounds/components known to persons skilled in the art. Collected samples may be stored at the site of collection at suitable temperature or they may be transported to a local or external laboratory for preparation.

The sample may be processed using various conventional techniques in order to obtain an optimal signal from any aggressive or metastatic cancer cells present in the sample. Processing the sample may include, but is not limited to, filtration, precipitation, immunoprecipitation, flow-sorting, lyzing, certifugation, cooling, freezing, heating or any other methods known to a person skilled in the art. Preferably, the sample is treated to allow optimal detection of podocalyxin and/or TRA molecules. This is, for example, performed by treating the sample in a manner that allows the molecules/cells of the sample to remain intact and, to the extent possible, also retain their original morphology.

The sample may be analyzed for the presence of biomarkers for the detection of podocalyxin and/or TRA molecules and aggressive or metastatic cancer cells using a variety of analyses. These analyses include immunoassays, immunostaining, immunofluorescence, immunohistochemistry, direct IHC, indirect IHC, immunocytochemistry, in-situ hybridization, fluorescent ISH, FISH in suspension, western blot, flow cytometry, fluorescence-activated cell sorting, imageStream, turtle probes, target primed rolling circle PRINS, luminex assay, polymerase chain reaction, mass spectrometry and the like. The analysis of the biological sample is performed in a specific manner to reduce the risk of obtaining false positive or false negative results.

In accordance with certain embodiments, analysis results showing positive reactivity is indicative of the presence of podocalyxin and/or TRA molecules in the sample being analyzed and little or no reactivity is indicative of the absence of podocalyxin and/or TRA molecules in the sample being analyzed.

The aggressive and metastatic cancer cells can be in any organ or tissue in the body. For example, and without limitation, the aggressive and metastatic cancer cells can be in the prostate, ovary, colon, breast, and testis. Further, the aggressive and metastatic cancer cells can include carcinomas that metastasize from other cancer tumors.

Furthermore, immunostaining and/or enzymatic assays for the detection of the at least one biochemical marker can be used during the screening for cancer cells in the biological sample.

Podocalyxin and TRA are embryonic in nature and are markers of human embryonic stem cells. As previously indicated, there may be low levels of podocalyxin and TRA in a healthy individual due to the presence of podocalyxin and TRA on normal stem cells such as embryonic stem cells. However, in a disease state, the levels of podocalyxin and TRA may be increased as compared to the levels present in the healthy/normal body. Further, it is contemplated and expected that the levels of podocalyxin and TRA in the samples obtained from patients with metastatic cancer will exceed the levels of podocalyxin and TRA in the samples obtained from patients with localized cancer.

In accordance with the invention, in certain embodiments, the level of podocalyxin and/or TRA in a biological sample obtained from a cancer-containing patient can be compared to the level of podocalyxin and/or TRA in a control sample obtained from a non-cancer-containing patient. In certain other embodiments, the level of podocalyxin and/or TRA in a biological sample obtained from a cancer-containing patient can be compared to the level of podocalyxin and/or TRA in another, e.g., earlier obtained, biological sample from the cancer-containing patient. If the level of podocalyxin and/or TRA in the biological sample from the cancer-containing patient is measurably higher or greater than the level of podocalyxin and/or TRA in the control sample or earlier obtained sample, then there is a diagnosis of the presence of podocalyxin and/or TRA in the biological sample from the cancer-containing patient. Correspondingly, there may be a diagnosis of aggressive and/or metastatic cancer cells in the cancer-containing patient. Further, there may be a recommendation of an aggressive treatment regimen for the cancer-containing patient. If the level of podocalyxin and/or TRA in the biological sample from the cancer-containing patient is measurably less than or essentially equal to the level of podocalyxin and/or TRA in the control sample or the earlier obtained sample, then there is a diagnosis of the absence of podocalyxin and/or TRA in the biological sample from the cancer-containing patient. Correspondingly, there may be a diagnosis of a lack of aggressive and/or metastatic cancer cells in the cancer-containing patient. Further, there may be a recommendation of a non-aggressive treatment regimen for the cancer-containing patient.

In other embodiments, the presence or absence of aggressive cancer may be determined based on comparison of a biological sample to a threshold value. There may be a threshold level of podocalyxin and/or TRA such that when the level of podocalyxin and/or TRA molecules measured in the biological sample is essentially equal to or measurably higher or greater than the threshold level, there is a diagnosis of the presence of podocalyxin and/or TRA in the biological sample from the cancer-containing patient. Correspondingly, there may be a diagnosis of aggressive and/or metastatic cancer cells in the cancer-containing patient. Further, there may be a recommendation of an aggressive treatment regimen for the cancer-containing patient. When the level of podocalyxin and/or TRA molecules measured in the biological sample is measurably less than the threshold level, there is a diagnosis of the absence of podocalyxin and/or TRA in the biological sample from the cancer-containing patient. Correspondingly, there may be a diagnosis of a lack of aggressive and/or metastatic cancer cells in the cancer-containing patient. Further, there may be a recommendation of a non-aggressive treatment regimen for the cancer-containing patient.

In accordance with the invention, in certain embodiments, a threshold level of podocalyxin and/or TRA or a threshold range of levels of podocalyxin and/or TRA found in normal, healthy patients may be determined by obtaining biological samples from these patients and measuring the level of podocalyxin and/or TRA in these patients. Based on this data, a threshold level or threshold range can be established and employed to determine the presence or absence of aggressive and/or metastatic cancer. When the podocalyxin and/or TRA level in a biological sample from a cancer-containing patient is essentially equal to or above the threshold level/range of podocalyxin and/or TRA, a diagnosis of the presence of aggressive and/or metastatic cancer is made. When the podocalyxin and/or TRA level in a biological sample from a cancer-containing patient is below the threshold level/range of podocalyxin and/or TRA, a diagnosis of the absence of aggressive and/or metastatic cancer may be made.

In certain embodiments, in accordance with this invention, blood and/or seminal and/or urine samples are collected from healthy controls, patients with localized cancer and patients with metastatic cancer. The samples collected from these three groups of patients are measured and analyzed using standard methods and apparatus to determine the presence or absence or level of podocalyxin and/or TRA molecules using the anti-podocalyxin, TRA-1-60 and TRA-1-81 monoclonal antibodies. These antibodies are commercially available from numerous sources and are widely used by the human stem cell research community for the characterization of embryonic stem cells. The levels of podocalyxin and/or TRA in the samples of patients having cancer (localized and metastatic) are compared with each other, and also can be compared with the healthy control samples. Alternatively, the podocalyxin and/or TRA level in the samples can be compared to a threshold value or range (e.g., such that a level above or below the value or range represents the presence or absence of aggressive and/or metastatic cancer).

In certain embodiments, analyzing the biological samples includes the use of a binding material which is selected to interact with podocalyxin and/or TRA molecules. The binding material may be effective to separate and isolate the podocalyxin and/or TRA molecules from the biological sample. Suitable binding materials are known in the art or may be prepared. In accordance with certain embodiments, the binding material is an antibody.

The stem cell hypothesis of cancer states that cancer is a disease of normal adult stem cells. Another version of this hypothesis is aggressive cancer—lethal cancer that kills the patient—utilizes stem cell properties and mechanisms to enable it to metastasize throughout the body, survive and thrive during standard cancer treatments, and grow, and reseed tumor burden after treatments.

In certain embodiments, a determination of the presence of podocalyxin and/or TRA in a biological sample of a cancer-containing patient corresponds to the presence of podocalyxin-expressing and/or TRA-expressing cancer stem cells in the patient, a diagnosis of aggressive and/or metastatic cancer and a recommendation of aggressive treatment for the cancer-containing patient; and a determination of the absence of podocalyxin and/or TRA in a tumor sample of a cancer-containing patient corresponds to the absence of podocalyxin-expressing and/or TRA-expressing cancer stem cells in the patient, a diagnosis of non-aggressive and/or no metastatic cancer and a recommendation of less aggressive treatment for the cancer-containing patient. As previously described, aggressive or more aggressive treatment includes surgery, chemotherapy, radiation therapy or combinations thereof, and non-aggressive or less aggressive treatment includes active surveillance, monitoring and assessing the patient. Thus, reducing or precluding unnecessary treatments and the potential serious complications associated therewith.

In one embodiment, this invention is used for the detection and diagnosis of aggressive and/or metastatic prostate cancer in male patients. In other embodiments, this invention is used for the detection and diagnosis of other aggressive and/or metastatic cancers in both male and female patients.

In an aspect, the invention provides a device for detecting the presence or absence of podocalyxin and/or TRA molecules in a biological sample and a diagnosis of the presence or absence of aggressive and/or metastatic cancer stem cells in a patient. The device can include a tool for obtaining a blood-derived specimen or a seminal fluid specimen or a urine specimen from the patient. Suitable tools are well known in the art and can include conventional syringes. The device further includes a means or mechanism for detecting podocalyxin and/or TRA molecules and determining their presence or absence in the specimen. In certain embodiments, a detector means is employed to determine the presence or absence of anti-podocalyxin, TRA-1-60 and/or TRA-1-81 in the specimen. Furthermore, the device includes an indicator means or mechanism for converting and/or transmitting the result of the detector means, e.g., the presence or absence of anti-podocalyxin, TRA-1-60 and/or TRA-1-81 in the specimen, to a displayable output to be read and/or interpreted by the patient and/or a medical professional.

In certain embodiments, the device is a kit, e.g., test kit. The kit includes a device for obtaining a biological sample from a patient, a mechanism for evaluating the biological sample, and a means for interpreting the results of the evaluation to determine the presence of podocalyxin and/or TRA, and providing a diagnosis of aggressive or metastatic cancer cells in the patient. The kit can be employed by the patient to perform self-testing and diagnosis or by a third party, e.g., a physician, nurse or medical technician, to perform testing and diagnosis on another person. In certain embodiments, the kit may include a test strip, or similar configuration, having at least one region for testing for aggressive or metastatic cancer and may provide a visual indication, either with the naked eye or through a proper instrument, of whether there is the presence or absence of podocalyxin and/or TRA and, therefore, whether there is a positive or negative diagnosis of the presence of aggressive and/or metastatic cancer stem cells and thus, the presence or absence of aggressive and/or metastatic cancer. An indication as to the presence of podocalyxin and/or TRA and an indication as to the presence of aggressive or metastatic cancer may be provided in the same or in different regions on the test strip. It is contemplated that a sufficient change in the region(s) of the test strip will provide the presence of podocalyxin and/or TRA and aggressive or metastatic cancer. It is further contemplated that the test strip may be immersed in the biological sample or a drop or portion of the sample be applied to or deposited on the test strip. An adequate period of time may be needed to allow for a reaction to occur; e.g., a few seconds or a few minutes. In certain embodiments, the test strip region(s) involve a reaction with anti-podocalyxin, TRA-1-60 and/or TRA-1-81 monoclonal antibodies. The region(s) of the test strip can be compared with a chart, e.g., a color chart, to determine the presence, absence or level or reactivity of podocalyxin and/or TRA in the sample. For example, the chart can include various intensities of a color or multiple colors. The greater the color intensity may be correlated to the higher the presence or level or reactivity of podocalyxin and/or TRA in the sample. The comparison can be made by the naked eye or alternatively, can be read by an instrument. Further, the instrument may be connected to a display mechanism.

In certain embodiments, a TRA-1-60 and TRA-1-81 enzyme-linked immunosorbent assay (ELISA) may be developed as a kit to be used in clinical labs both in hospitals and independent commercial labs. The kit can include microtiter plates coated with a binding material, such as Bstrongomab antibody or a different antibody with similar properties. The binding material is effective to interact with the TRA-1-60 and TRA-1-81 and to bind these epitopes such as to separate, e.g., isolate, them from the biological specimen that is being tested. In addition, the kit may include one or more of the following: buffers; positive and negative TRA-1-60 and TRA-1-81 controls, TRA-1-60 and TRA-1-81 specific primary antibodies and conjugated secondary antibodies specific for the primary antibodies. The kit may be used to determine the levels of TRA-1-60 and TRA-1-81 molecules in the biological specimen of patients with cancer or suspected of having cancer.

The concentrations of TRA-1-60 and TRA-1-81 molecules in a patient specimen may be measured by other methods instead of an ELISA. For example, they can be measured by western blotting techniques, or by mass spectrometry or NMR or any other method which is generally capable of measuring the concentration of a specific molecule in biological liquid or fluid, such as blood, urine or seminal fluid.

It will be appreciated that kits in accordance with the invention may include alternate designs and configurations.

The devices and methods of the invention may be used to determine the presence or absence or level of cancer stem cells or cancer cells having stem cell-like properties, how aggressive is the cancer in a patient, the likelihood that localized cancer will metastasize or develop into aggressive cancer, and the appropriate treatment regimen. The results obtained by the devices and methods of the invention provide a source of information and data for consideration in making these determinations. The results can be used alone or in combination with other cancer-related information and data that may be available.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof. Furthermore, the following examples are meant to be illustrative of certain embodiments of the invention and are not limiting as to the scope of the invention.

### EXAMPLES

### Example 1

The following tests were conducted to demonstrate that the human pluripotent stem cell marker, TRA-1-60, is detected in serum from a patient with metastatic prostate carcinoma but is not detected in serum from a patient with localized prostate carcinoma.

From each of a patient with metastatic prostate cancer and a patient with localized prostate cancer was collected 1 ml of serum (serum obtained from Folio Biosciences, Powell, Ohio). These samples were incubated with Separose 4B beads conjugate with Peanut Agglutinin at 5 mg/ml of beads for 1 hour at room temperature. The beads were then centrifuged and, the serum was removed and washed three times with Phosphate Buffer saline (PBS). The beads were centrifuged a final time and re-suspended in 100 ul of SDS-PAGE loading buffer and heated at 70 C.° for 5 minutes to release proteins bound to the PNA sepharose beads. Protein samples were separated by 7.5% SDS-PAGE protein gels and transferred for 1 hour at 300 mVolts to nitrocellulose paper followed by blocking in 5% nonfat milk/PBS with 0.1% Tween 20. After blocking, the blot was incubated with TRA-1-60 antibody at 1 ug/ml in PBS-0.1% Tween for one hour at room temperature, followed with secondary goat anti-mouse IgM peroxidase-conjugated antibody. The blots were washed and developed with ECL and x-ray film.

The developed film showed the presence of a TRA-1-60 specific 200 kilodalton diffuse band from the metastatic serum sample but no band was detected from the localized prostate cancer serum sample.

As controls for the experiment, protein extracts from cancer stem cells, TERA-1 and NCCIT, were loaded onto the gels. Both samples showed positive TRA-1-60 bands on the film.

The results of this experiment demonstrate that TRA-1-60 is a serum marker for metastatic prostate cancer. TRA-1-60 was not detected in the serum of a patient with localized prostate cancer. Thus, TRA-1-60 is a biomarker which can be used as a test to determine whether a patient with prostate cancer has aggressive and lethal metastatic cancer, or the less aggressive and non-lethal form of prostate cancer.

### Example 2

Plasma and serum samples from blood of patients with metastatic prostate, breast, lung, colon and kidney cancers along with plasma and serum samples of blood from healthy individuals with no evidence of cancer or from patients with localized but not metastatic cancers were obtained from Folio Biosciences (1476 Manning Parkway, Powell, Ohio, 43065) a commercial provider of human biospecimens. In addition, plasma and serum samples from blood of patients with metastatic prostate cancer and patients with localized prostate cancer and no evidence of metastatic disease were also obtained from Karmanos Cancer Institute, Detroit, Mich. as part of a collaborative research study of an IRB approved pilot clinical diagnostic study designed to measure the levels of the pluripotent stem cell markers TRA-1-60 and TRA-1-81 in patients with prostate cancer.

To measure the relative levels of the TRA-1-60 and TRA-1-81 pluripotent stem cell markers in plasma and serum blood samples, an ELISA (enzyme-linked immunosorbent assay) was developed with a “capture’ antibody, Bstrongomab 9A, which is a binding material and was used to isolate the TRA-1-60 and TRA-1-81 carbohydrate molecules from the plasma and serum samples. That is, the Bstrongomab 9A interacted with the TRA-1-60 and TRA-1-81 carbohydrate molecules to bind with these epitopes such that they were separated from the plasma and serum samples. Bstrongomab 9A is a mouse monoclonal, IgG antibody that targets a high molecular weight protein (molecular weight 200 and 250 kDa) on pluripotent cancer cells such as TERA1 (ATCC, 10801 University Blvd. Manassas, Va. 20110, Lot number HTB-105) and NCCIT (ATCC, University Blvd. Manassas, Va. 20110, Lot number CRL-2073) mammalian pluripotent embryonal carcinoma cell lines. The Bstrongomab 9A mouse hybridoma cell line was developed by CureMeta (Boston, Mass.) using TERA1 whole cells as the immune antigen. Bstrongomab 9A antibody was purified from hybridoma supernatants using Hi Trap Protein G HP column (GE Healthcare, Catalog number 17-0404-01) following manufacturer's protocol. Purified Bstrongomab 9A was desalted into PBS using Zeba Spin column (Thermo Fisher, catalog number PI-89892). Bstrongomab 9A concentration was determined by measuring absorbance at 280 nm and using the extinction coefficient 1.36.

The TRA-1-60 and TRA-1-81 ELISA was designed to measure the levels of the TRA-1-81 and TRA-1-60 pluripotent carbohydrate stem cell markers whether the carbohydrate molecule was present alone as a soluble molecule in blood, or the carbohydrate structure was attached to the stem cell protein marker podocalyxin in blood, or if the carbohydrate structure, with or without podocalyxin, were bound to a common blood protein ‘carrier’ molecule such as albumin. In addition, the TRA-1-81 and TRA-1-60 stem cell markers can be separate and distinct carbohydrate molecules or can be part of the same carbohydrate molecule present in blood.

### TRA-1-60 and TRA-1-81 ELISA Assay Procedure

White opaque 96-well half-area high binding microtiter plates (USA Scientific Inc, Ocala, Fla., catalog number 675074) were coated with Bstrongomab 9A antibody by incubating each well with 15 microliter of phosphate buffer solution (Fisher Scientific, Waltham, Mass., catalog number BP2944-100) containing the Bstrongomab 9A antibody at concentration of 15 microgram/milliliter overnight at 4 C.°. The microtiter plates were washed three times with 175 microliters of phosphate buffer solution prior to the addition of 175 microliters of SuperBlock (Fisher Scientific, Waltham, Mass.). All incubations of the microtiter plates in this ELISA assay including blocking was performed using a Boekel Scientific Jitterbug (model 130000E5) at a temperature of 37 C.° for a duration of 30 minutes. Once blocked, the microplates were emptied with a flicking motion above a sink Serum and/or plasma samples were added in columns 1 through 9 and columns 10, 11 and 12 were reserved for positive and negative controls. A multi electronic pipette (ErgoOne, USA Scientific, Ocala, Fla.) was used to fill each well with 15 microliters of sample, which is the recommended working volume for this plate. The negative controls consisted of PBS with 0.1 tween (Sigma, Louis, Mo.) and 5% powder milk. The positive controls solutions were made with same reagents as negative control except for an addition of cell lysate produced from TERA-1 cell line at total protein concentration (Protein concentration measured by BSA assay: ThermoScientific, Rockland, Il, and read on plate reader: Molecular Devices, Sunnyvale, Calif.) of 1 microgram/mL or 0.1 microgram/mL TERA-1 protein lysates were made by lysing freshly harvested TERA-1 cells in three volumes of lysis buffer that contained PBS with 1% Chaps (Sigma, St Louis, Mo.) and protease inhibitors used at concentrations according to manufacturer's guidelines (Sigma, St Louis, Mo., catalog number P8849), for one hour at room temperature followed by centrifugation at 15,000×g's to remove insoluble cellular debris. After a 30 minute incubation of the samples and the controls, the microplate was washed 3 times with 175 microliters of PBS with 0.1% Tween using a manual multi channel pipette. At this point in the assay, the primary antibody was added. If the assay was a TRA-1-60 ELISA, every well received 15 microliter of the primary antibody TRA-1-60 (Life Technologies, Grand Island, N.Y., catalog number 411000) which was prepared as a 1 to 250 parts dilution in buffer prepared according to the recipe of the negative control. If the assay was a TRA-1-81 ELISA, every well received 15 microliter of the primary antibody TRA-1-81 (Life Technologies, Grand Island, N.Y., catalog number 411100) which was prepared as a 1 to 250 parts dilution in buffer prepared according to the recipe of the negative control. After primary antibody incubation, all the wells of the microplate were washed three times with 175 microliters of PBS with 0.1% tween. The secondary anti-mouse IGM HRP conjugated antibody (EMD Millipore, Billerica, Mass., catalog number AP500P) was diluted 1 to 500 parts in buffer and 15 microliters of this solution was added to all the wells. After one hour incubation, the microtiter plate was washed five times with 175 microliters PBS with 0.1% Tween and between every other wash the plates were allowed to incubate from 3 to 5 minutes. A final wash was with PBS without any Tween was performed before adding 15 microliter of the Supersignal substrate (Fisher Scientific Rockford, Il, catalog number 37074). The microtiter plates were measured in luminescence mode with a Filtermax F5 plate reader (Molecular Devices, Sunnyvale, Calif.) using capture duration of each well of 400 milliseconds.

To perform data analysis for an ELISA, a raw data file was exported from the plate reader software containing the luminescent readings for each well to a memory stick. The stick was then physically moved to a chrome computer and uploaded in the Google spreadsheet application. The luminescent readings of all wells were normalized using a spreadsheet template where the median of the 8 negative controls was set to 0 and the median of the 8 positive controls wells at the 1 microgram/mL protein concentration was set to 100 units of normalized luminescence (NL). The higher the NL number, the greater the amount of TRA-1-60 or TRA-1-81 in the blood sample. The additional 8 positive controls at a concentration of 0.1 microgram was used as quality control parameter. This normalization procedure allowed for comparison of samples between different microtiter plates. The normalized luminescence (NL) values are reported as a range because the ELISA was done more than one time for almost all samples.

Results of TRA-1-60 ELISA Analysis of Plasma Samples from Patients with Prostate Cancer Obtained from Karmanos Cancer Institute

A sandwich ELISA assay was used to detect the level of TRA-1-60 antigen in plasma samples from eight patients with localized prostate cancer (samples K21-K28) and eight patients with metastatic prostate cancer (samples K11-K18). The plasma samples were kept at −80 and thawed just prior to testing. All samples had been tested at sample volume of 15 ul. Plasma samples were distributed into 8 wells on two separate plates ELISA plates and were processed in parallel. The results from prostate cancer plasma samples showed that two patients (K17 and K18) diagnosed with metastatic cancer had a high NL ELISA signal (sample K17 range of NL values 47 to 84; sample K18 range of NL values 9 to 26) that was at least ten times higher than any patient diagnosed with the localized prostate cancer (samples K21 to K28, range of median NL values −3 to 2). Two other plasma samples of patients with metastatic prostate cancer (samples K12 and K13) had median level of over 3 (sample K12 range of NL 2 to 4; sample K13 range of NL values 1 to 4). The plasma of the other four patients with metastatic prostate cancer (samples K11, K14, K15, and K16 had a range of median NL values of −1 to 0). These results showed that TRA-1-60 pluripotent stem cell marker was detected in four of eight prostate cancer patients that had advanced metastatic disease but was not detected in eight of eight patients with local non-advanced disease.

Results of TRA-1-60 ELISA Analysis of Serum Samples of Patients with Prostate Cancer

A TRA-1-60 ELISA assay was used to detect the level of TRA-1-60 antigen in serum samples from three patients with localized prostate cancer (samples F21-F23) and three patients with metastatic prostate cancer (samples F11-F13). The plasma samples were kept at −80 and thawed just prior to testing. Serum from three patients diagnosed with metastatic prostate cancer obtained from Folio had the following ranges of NL values; F11: 20 to 26, F12: −1 to 3, F13: −2 to 1, while the three serum samples of patients with localized prostate cancer had NL value ranges between −3 to 0. These results showed that one of three serum samples of patients with metastatic prostate cancer had detected levels of the TRA-1-60 pluripotent stem cell marker in their blood while no detectable levels were detected in three of three patients that had local prostate cancer disease.

Results of TRA-1-60 ELISA Analysis of Serum Samples of Patients with Breast Cancer

A TRA-1-60 ELISA assay was used to detect the level of TRA-1-60 antigen in serum samples from three patients with localized breast cancer (samples F41-F43) and six patients with metastatic breast cancer (samples F31-F36). The plasma samples were kept at −80 and thawed just prior to testing. Serum from six patients with metastatic breast cancer obtained from Folio had the following ranges of NL values; F31: 20 to 38, F32: 7 to 12, F33: 0 to 3, F34: −2 to 3, F35: −3 to 0, F36: −3 to −2. The serum samples of the three patients with localized breast cancer had an average of range of NL values of −2 to 1. These results showed that three of six patients with metastatic breast cancer had detectable levels of the TRA-1-60 pluripotent stem cell marker in their blood while no detectable levels of TRA-1-60 was detected in three of three patients that had localized breast cancer disease.

Results of TRA-1-60 ELISA Analysis of Serum or Plasma Samples of Patients with Metastatic Lung, Colorectal or Kidney Cancer

To test other kinds of advance cancers for the presence of TRA-1-60 in the blood we obtained plasma or serum samples from Folio Biosciences of patients with metastatic cancers. As a control, we also obtained from Folio Biosciences ten serum specimens from healthy volunteers with no detectable presence of cancer. The average range of NL values for the ten control samples (F91-F99) was −2 to 2. Three plasma samples (F51-F53) from patients with metastatic lung cancer had the following ranges of NL values: F51: 44 to 54, F52: 100 to 118, F53: 2 to 4. Three serum samples (F61-63) from patients with metastatic colorectal cancer had the following TRA-1-60 NL values; F61: 46 to 94, F62: 1 to 4, F-63: 0 to 2. Two serum samples (F71-72) from patients with metastatic kidney cancer had NL values of: F71: 164 (a single value because it was ran only once); F72: −3 to −4. These results show that in addition to metastatic breast and prostate cancer, TRA-1-60 was detected in the blood of patients with three other types of metastatic cancers including kidney, colorectal and lung cancers.

Results of TRA-1-81 ELISA Analysis of Serum and Plasma Samples of Patients with Metastatic Prostate, Breast, Lung, Kidney and Colorectal Cancers

Because the TRA-1-81 marker is highly similar to the TRA-1-60 marker (the molecular epitope that defines both markers exist on the same pluripotent carbohydrate structure present on all human pluripotent stem cells), the results of TRA-1-81 and TRA-1-60 ELISA analysis of the same blood samples should be highly similar. A TRA-1-81 ELISA was done on one metastatic prostate cancer plasma samples (F11), one metastatic breast cancer serum samples (F31), three plasma metastatic lung cancer samples (F51-F53), three serum samples (F61-63) and two metastatic kidney serum samples (F71, F72). In addition, ten normal serum and plasma samples (F91-F99) were also analyzed. Results of the TRA-1-81 ELISA showed the following TRA-1-81 NL values: the average range of NL values for the ten control samples (F91-F99) was −3 to 3 and the NL values for the metastatic cancer samples were: metastatic prostate cancer, F11: 18 to 21; metastatic breast cancer, F31: 40-55; metastatic lung cancer, F51: 76 to 126, F52: 79-94, F53: −9 to −11; metastatic colorectal cancer, F61: 32 to 130, F62: −8 to 0, F63; −2 to; metastatic kidney cancer, F71: 164 (single value—sample only ran once), F72: 1 to 2. These TRA-1-81 ELISA results show that the pluripotent stem cell marker TRA-1-81 was detected in the blood of patients with metastatic cancer while TRA-1-81 was not detected in normal control blood. Also, these results show that the TRA-1-60 ELISA and TRA-1-81 ELISA produced similar values for the same blood sample which was expected.

We claim:

1. A method of detecting the presence of podocalyxin and/or TRA molecules in a first patient, comprising:
obtaining a first biological sample from the first patient, the first sample selected from the group consisting of a blood-derived specimen, a seminal fluid specimen, a urine specimen, and combinations thereof;
testing the first biological sample for the presence of podocalyxin and/or TRA molecules; and
determining the presence or absence of podocalyxin and/or TRA molecules in the first biological sample.

2. The method of claim 1, wherein the podocalyxin and/or TRA molecules are selected from the group consisting of anti-podocalyxin, TRA-1-60, TRA-1-81, and mixtures thereof.

3. The method of claim 1, wherein the first biological sample is compared to a second biological sample selected from the group consisting of an earlier obtained biological sample from the first patient or a control biological sample obtained from a second patient without cancer.

4. The method of claim 1, wherein the first biological sample is compared to a predetermined threshold value.

5. The method of claim 1, further comprising providing a medical diagnosis of a presence of metastatic cancer cells in the first patient based on the presence of the podocalyxin and/or TRA molecules in the first biological sample.

6. The method of claim 1, further comprising providing a medical diagnosis of an absence of metastatic cancer in the patient based on the absence of the podocalyxin and/or TRA molecules in the sample.

7. The method of claim 3, further comprising providing a medical diagnosis of a presence of metastatic cancer cells in the first patient based on a level of the podocalyxin and/or TRA molecules in the first biological sample being measurably greater than a level of podocalyxin and/or TRA molecules in the second biological sample.

8. The method of claim 3, further comprising providing a medical diagnosis of an absence of metastatic cancer cells in the first patient based on a level of the podocalyxin and/or TRA molecules in the first biological sample being essentially equal to or measurably less than a level of podocalyxin and/or TRA molecules in the second biological sample.

9. The method of claim 4, further comprising providing a medical diagnosis of a presence of metastatic cancer cells in the first patient based on a level of the podocalyxin and/or TRA molecules in the first biological sample being essentially equal to or measurably greater than the threshold value.

10. The method of claim 4, further comprising providing a medical diagnosis of an absence of metastatic cancer cells in the first patient based on a level of the podocalyxin and/or TRA molecules in the first biological sample being measurably less than the threshold value.

11. A method of detecting and diagnosing aggressive cancer stem cells in a patient, comprising:
obtaining a biological sample from the patient, the biological sample selected from the group consisting of a blood-derived specimen, urine specimen and combinations thereof;
analyzing the biological sample for a presence of podocalyxin and/or TRA molecules selected from the group consisting of anti-podocalyxin, TRA-1-60, TRA-1-81 and mixtures thereof;
determining the presence or an absence of the podocalyxin and/or TRA molecules in the biological sample;
providing a diagnosis of the presence of aggressive cancer stem cells in the patient based on the presence of the podocalyxin and/or TRA molecules in the biological sample; and
providing a diagnosis of the absence of aggressive cancer stem cells in the patient based on the absence of the podocalyxin and/or TRA molecules in the biological sample.

12. The method of claim 11, wherein the analyzing of the biological sample comprises employing a binding material selected to interact with the podocalyxin and/or TRA molecules in the biological sample.

13. The method of claim 12, wherein the binding material is an antibody.

14. A device for detecting and diagnosing aggressive cancer stem cells in a patient; the device comprising:
a tool for obtaining a biological sample from the patient, the biological sample selected from the group consisting of a blood-derived specimen, a seminal fluid specimen, a urine specimen and combinations thereof;
a detector means for determining the presence or absence of podocalyxin and/or TRA molecules selected from the group consisting of anti-podocalyxin, TRA-1-60, TRA-1-81 and mixtures thereof; and
an indicator means to display the presence of the podocalyxin and/or TRA molecules in the biological sample.

15. The device of claim 14, wherein the device is a test kit, comprising:
an enzyme-linked immunosorbent assay; and
at least one testing plate coated with a binding material selected to interact with anti-podocalyxin, TRA-1-60 or TRA-1-81 and to separate the podocalyxin and/or TRA molecules from the biological sample.
